(19) United States
(12) Patent Application Publication
(10) Pub. No.: US 2024/0315759 A1
(43) Pub. Date: Sep. 26, 2024

(54) SURGICAL INSTRUMENT

(71) Applicant: Covidien LP, Mansfield, MA (US)

(72) Inventors: Robert F. McCullough, JR., Boulder, CO (US); Ryan C. Artale, Crested Butte, CO (US); Robert M. Sharp, Boulder, CO (US); Tony Moua, Thornton, CO (US); Mark J. Huseman, Broomfield, CO (US); Audrey Earnshaw, Erie, CO (US)

(21) Appl. No.: 18/731,513

(22) Filed: Jun. 3, 2024

Related U.S. Application Data
(63) Continuation of application No. 16/893,508, filed on Jun. 5, 2020, now Pat. No. 12,011,208.
Application No. 16/893,508 is a continuation of application No. 15/458,228, filed on Mar. 14, 2017, now Pat. No. 10,687,886.
Application No. 15/458,228 is a continuation of application No. 14/196,066, filed on Mar. 4, 2014, now Pat. No. 9,655,673.
(60) Provisional application No. 61/776,136, filed on Mar. 11, 2013.

(51) Int. Cl. A61B 18/14; A61B 17/29; A61B 18/00; A61B 90/00

(52) U.S. Cl. CPC A61B 18/1445; A61B 2017/292; A61B 2018/00601; A61B 2018/0063; A61B 2018/00928; A61B 2018/1455; A61B 2090/031

(57) ABSTRACT

An electrosurgical instrument includes a housing including an elongated shaft. A stationary actuation member is axially disposed within the elongated shaft and includes a cam pin. An actuating mechanism is operably coupled to the elongated shaft and is moveable relative to the housing to selectively cause movement of the elongated shaft. An end effector includes a pair of opposing first and second jaw members operably coupled about a common pivot. One or both of the jaw members includes a camming slot configured to engage the cam pin. Each jaw member includes an electrically conductive tissue sealing surface adapted to connect to a source of electrosurgical energy. A knife blade is supported in the elongated shaft and moveable in a longitudinal direction to cut tissue disposed between the jaw members. A switch is configured to be engaged by the actuating mechanism to initiate delivery of electrosurgical energy to tissue.

### CROSS-REFERENCE TO RELATED APPLICATIONS

This application is a continuation of U.S. patent application Ser. No. 16/893,508, filed on Jun. 5, 2020, now U.S. Pat. No. 12,011,208, which is a continuation of U.S. patent application Ser. No. 15/458,228, filed on Mar. 14, 2017, now U.S. Pat. No. 10,687,886, which is a continuation of U.S. patent application Ser. No. 14/196,066, filed on Mar. 4, 2014, now U.S. Pat. No. 9,655,673, which claims the benefit of the filing date of provisional U.S. Patent Application No. 61/776,136, filed on Mar. 11, 2013.

### INTRODUCTION

The present disclosure relates generally to the field of surgical instruments. In particular, the disclosure relates to an endoscopic electrosurgical forceps that is economical to manufacture and is capable of sealing and cutting relatively large tissue structures.

### BACKGROUND

Instruments such as electrosurgical forceps are commonly used in open and endoscopic surgical procedures to coagulate, cauterize and seal tissue. Such forceps typically include a pair of jaw members that can be controlled by a surgeon to grasp targeted tissue, such as, e.g., a blood vessel. The jaw members may be approximated to apply a mechanical clamping force to the tissue, and are associated with at least one electrode to permit the delivery of electrosurgical energy to the tissue. The combination of the mechanical clamping force and the electrosurgical energy has been demonstrated to join adjacent layers of tissue captured between the jaw members. When the adjacent layers of tissue include the walls of a blood vessel, sealing the tissue may result in hemostasis, which may facilitate the transection of the sealed tissue. A detailed discussion of the use of an electrosurgical forceps may be found in U.S. Pat. No. 7,255,697 to Dycus et al.

A bipolar electrosurgical forceps typically includes opposed electrodes disposed on clamping faces of the jaw members. The electrodes are charged to opposite electrical potentials such that an electrosurgical current may be selectively transferred through tissue grasped between the electrodes. To effect a proper seal, particularly in relatively large vessels, two predominant mechanical parameters must be accurately controlled; the pressure applied to the vessel, and the gap distance established between the electrodes.

Both the pressure and gap distance influence the effectiveness of the resultant tissue seal. If an adequate gap distance is not maintained, there is a possibility that the opposed electrodes will contact one another, which may cause a short circuit and prevent energy from being transferred through the tissue. Also, if too low a force is applied the tissue may have a tendency to move before an adequate seal can be generated. The thickness of a typical effective tissue seal is optimally between about 0.001 and about 0.006 inches. Below this range, the seal may shred or tear and above this range the vessel walls may not be effectively joined. Closure pressures for sealing large tissue structures preferably fall within the range of about 3 kg/cm² to about 16 kg/cm².

### SUMMARY

The present disclosure relates to an electrosurgical apparatus and methods for performing electrosurgical procedures. More particularly, the present disclosure relates to electrosurgically sealing tissue.

The present disclosure describes an electrosurgical instrument for treating tissue that is economical to manufacture and is capable of sealing and cutting relatively large tissue structures.

The electrosurgical instrument includes a housing including an elongated shaft having distal and proximal portions. The proximal portion is coupled to the housing. As is traditional, the term “distal” refers herein to an end of the apparatus that is farther from an operator, and the term “proximal” refers herein to the end of the electrosurgical forceps that is closer to the operator.

The elongated shaft defines a longitudinal axis. A stationary actuation member is axially disposed within the elongated shaft and includes a cam pin mechanically coupled to a distal end thereof. An actuating mechanism is operably coupled to the proximal portion of the elongated shaft and is moveable relative to the housing to selectively cause movement of the elongated shaft along the longitudinal axis relative to the stationary actuation member. An end effector includes a pair of opposing first and second jaw members operably coupled about a common pivot such that at least one of the jaw members is movable relative to the other jaw member from a first position wherein the jaw members are disposed in spaced relation relative to one another to a second position wherein the jaw members cooperate to grasp tissue therebetween. At least one of the first and second jaw members includes a camming slot configured to engage the cam pin to move the at least one movable jaw member between the first position and the second position upon movement of the elongated shaft along the longitudinal axis. Each jaw member includes an electrically conductive tissue sealing surface. Each tissue sealing surface is adapted to connect to a source of electrosurgical energy for conducting electrosurgical energy through tissue grasped therebetween to effect a tissue seal. A knife blade is supported in the elongated shaft and is moveable in a longitudinal direction through a knife channel defined along a length of at least one of the jaw members to cut tissue disposed between the jaw members. A switch is supported by the housing and is configured to be engaged by the actuating mechanism to initiate delivery of electrosurgical energy from the electrosurgical energy source to the end effector to treat tissue.

Additionally or alternatively, the switch is operably coupled to a depressible button extending from the housing. The button is configured to be selectively engaged by the actuating mechanism to activate the switch.

Additionally or alternatively, the second jaw member is mechanically coupled to a distal end of the elongated shaft and the first jaw member is configured to move relative to the second jaw member.

Additionally or alternatively, the stationary actuation member includes a longitudinal recess formed along a length thereof. The longitudinal recess is configured to permit movement of the pivot pin in a longitudinal direction upon movement of the elongated shaft.

Additionally or alternatively, the actuation mechanism is configured to engage a mechanical interface disposed within the housing. The mechanical interface is configured to generate a response to engagement with the actuation mechanism upon movement thereof relative to the housing. The mechanical interface may be constructed of a plastic film or the mechanical interface may be constructed of sheet metal. The response may be tactile and/or audible and may correspond to the second position of at least one jaw member. Additionally or alternatively, the response may indicate a position of the actuation mechanism relative to the switch.

Additionally or alternatively, the actuation mechanism includes a handle moveable relative to the housing between a distal position to move at least one jaw member to the first position and a proximal position to move the at least one jaw member to the second position. The handle may engage the switch upon movement of the handle to the proximal position.

Additionally or alternatively, movement of the knife blade in a longitudinal direction is prevented when the handle is in the distal position.

Additionally or alternatively, at least one of the jaw members includes an insulator coupled thereto. The insulator may be configured to electrically insulate the electrically conductive tissue sealing surface from the jaw member. The insulator may form at least one knife blade guide configured to guide the knife into the knife channel.

Additionally or alternatively, the insulator is configured to control splay of at least one of the jaw members.

According to another aspect of the present disclosure, an electrosurgical instrument is provided. The electrosurgical instrument includes a housing and an elongated shaft coupled to the housing and defining a longitudinal axis. An actuating mechanism is operably coupled to the elongated shaft and moveable relative to the housing to selectively cause movement of the elongated shaft along the longitudinal axis. An end effector is supported by the elongated shaft and is adapted for treating tissue. The end effector includes first and second jaw members pivotally coupled to one another to move between open and closed configurations. Each of the jaw members includes a camming surface. A switch is supported by the housing and is configured to be engaged by the actuating mechanism to initiate treatment of tissue. A knife rod extends at least partially through the elongated shaft and is selectively movable in a longitudinal direction. A blade operably coupled to the knife rod is extendable through a knife channel defined along a length of at least one of the jaw members. An inner actuation member extends at least partially through the elongated shaft and the elongated shaft is selectively movable in a longitudinal direction with respect to the knife and with respect to the inner actuation member. The inner actuation member carries a cam pin positioned to engage the camming surface of each of the jaw members to induce the jaw members to move between the open and closed configurations.

According to another aspect of the present disclosure, an electrosurgical system for performing electrosurgery is provided. The electrosurgical system includes an electrosurgical generator configured to provide electrosurgical energy and an electrosurgical instrument. The electrosurgical instrument includes a housing including an elongated shaft having distal and proximal portions. The proximal portion is coupled to the housing. The elongated shaft defines a longitudinal axis. A stationary actuation member is axially disposed within the elongated shaft and includes a cam pin mechanically coupled to a distal end thereof. An actuating mechanism is operably coupled to the proximal portion of the elongated shaft and is moveable relative to the housing to selectively cause movement of the elongated shaft along the longitudinal axis relative to the stationary actuation member. An end effector includes a pair of opposing first and second jaw members operably coupled about a common pivot such that at least one of the jaw members is movable relative to the other jaw member from a first position wherein the jaw members are disposed in spaced relation relative to one another to a second position wherein the jaw members cooperate to grasp tissue therebetween. At least one of the first and second jaw members includes a camming slot configured to engage the cam pin to move the at least one movable jaw member between the first position and the second position upon movement of the elongated shaft along the longitudinal axis. Each jaw member includes an electrically conductive tissue scaling surface. Each tissue sealing surface is configured to connect to the electrosurgical generator for conducting electrosurgical energy through tissue grasped therebetween to effect a tissue seal. A knife blade is supported in the elongated shaft and is moveable in a longitudinal direction through a knife channel defined along a length of at least one of the jaw members to cut tissue disposed between the jaw members. A switch is supported by the housing and is configured to be engaged by the actuating mechanism to initiate delivery of electrosurgical energy from the electrosurgical generator to the end effector to treat tissue.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1 is a perspective view of an electrosurgical forceps according to an embodiment of the present disclosure including a housing, an elongated shaft, and an end effector;

FIG. 2A is an enlarged, perspective view of the end effector of FIG. 1 depicted with a pair of jaw members in an open configuration;

FIG. 2B is an enlarged, perspective view of the end effector of FIG. 1 depicted with the pair of jaw members in a closed configuration;

FIG. 3A is a perspective view of the end effector and elongated shaft of FIG. 1 with parts separated;

FIG. 3B is cross-sectional view taken along line 3B-3B of FIG. 3A showing a distal portion of the electrosurgical forceps of FIG. 1 depicting a tube guide;

FIG. 3C is a perspective view of another knife blade and knife bar configuration for use with the end effector and elongated shaft of FIG. 1;

FIG. 3D is an enlarged, perspective view of the area of detail indicated in FIG. 3C;

FIG. 3E is a perspective view of a portion of another elongated shaft, similar to the elongated shaft of FIG. 1, configured for use with the end effector of FIG. 1;

FIG. 4 is a proximally-facing, perspective view of a rotation knob depicting a passageway for receiving the elongated shaft of FIG. 1;

FIG. 5 is a cross-sectional, perspective view of the end effector of FIG. 1;

FIG. 6 is a partial, proximal-facing perspective view of a distal portion of a jaw actuation mechanism of the end effector of FIG. 1;

FIG. 7 is a partial, distal-facing perspective view of distal portion of a knife actuation mechanism of the end effector of FIG. 1;

FIG. 8 is a perspective view of a lower jaw member of the end effector of FIG. 1;

FIG. 8A is an enlarged, perspective view of a portion of another lower jaw member, similar to the lower jaw member of FIG. 8, configured for use with the end effector of FIG. 1;

FIG. 9 is a cross-sectional, perspective view of the lower jaw member of FIG. 8;

FIG. 10 is a perspective view of a proximal portion of the instrument of FIG. 1 with a portion of the housing removed revealing internal components;

FIG. 10A is a cross-sectional view of another switch and activation button configured for use with the instrument of FIG. 1;

FIG. 11 is a partial, side view of a proximal portion of the instrument of FIG. 1;

FIG. 12A is a perspective view of a proximal portion of the knife actuation mechanism of the end effector of FIG. 1;

FIG. 12B is a cross-sectional, side view of a knife collar of the knife actuation mechanism of the end effector of FIG. 1;

FIG. 13A is a side view of the proximal portion of the instrument of FIG. 10 depicting a movable handle in a separated position with respect to a stationary handle, which corresponds to the open configuration of the end effector depicted in FIG. 2A, and a knife trigger in a separated configuration with respect to the stationary handle, which corresponds to an un-actuated or proximal configuration of a knife with respect to the jaw members;

FIG. 13B is a side view of the proximal portion of the instrument of FIG. 10 depicting the movable handle in an intermediate position with respect to the stationary handle, which corresponds to a first closed configuration of the end effector wherein the jaw members encounter one another;

FIG. 13C is a side view of the proximal portion of the instrument of FIG. 10 depicting the movable handle in an approximated configuration with respect to the stationary handle, which corresponds to a second closed configuration of the end effector wherein the jaw members apply an appropriate pressure to generate a tissue seal; and

FIG. 13D is a side view of the proximal portion of the instrument of FIG. 10 depicting the knife trigger in an actuated configuration, which corresponds to an actuated or distal position of the knife with respect to the jaw members.

### DETAILED DESCRIPTION

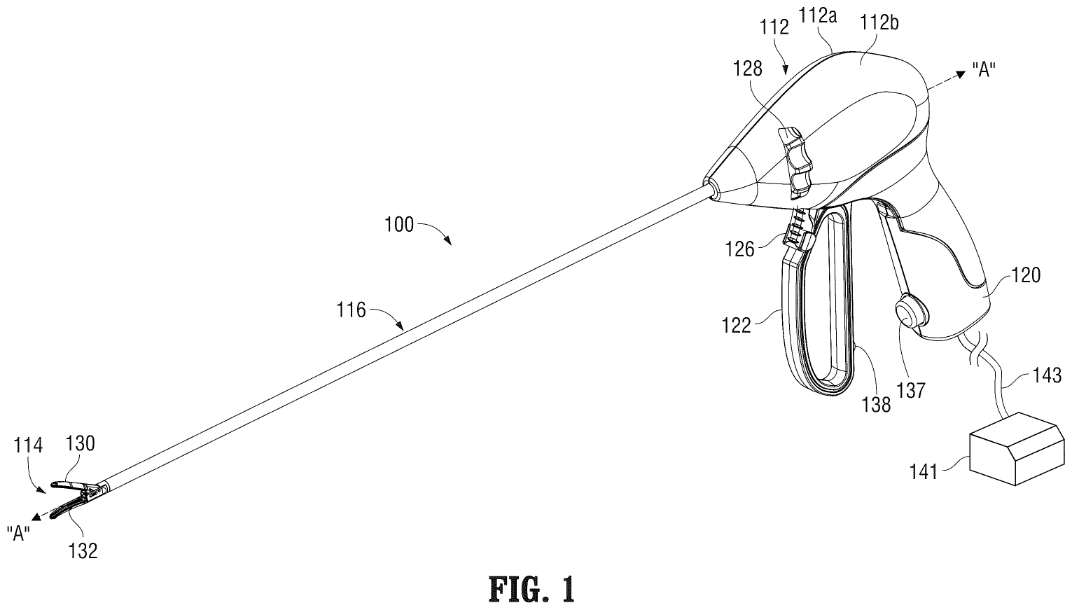

Referring initially to FIG. 1, an electrosurgical forceps 100 generally includes a housing 112 that supports various actuators thereon for remotely controlling an end effector 114 through an elongated shaft 116. Although this configuration is typically associated with instruments for use in laparoscopic or endoscopic surgical procedures, various aspects of the present disclosure may be practiced with traditional open instruments and in connection with endoluminal procedures as well. The housing 112 is constructed of a left housing half 112a and a right housing half 112b. The left and right designation of the housing halves 112a, 112b refer to the respective directions as perceived by an operator using the forceps 100. The housing halves 112a, 112b may be constructed of sturdy plastic, and may be joined to one another by adhesives, ultrasonic welding or other suitable assembly methods.

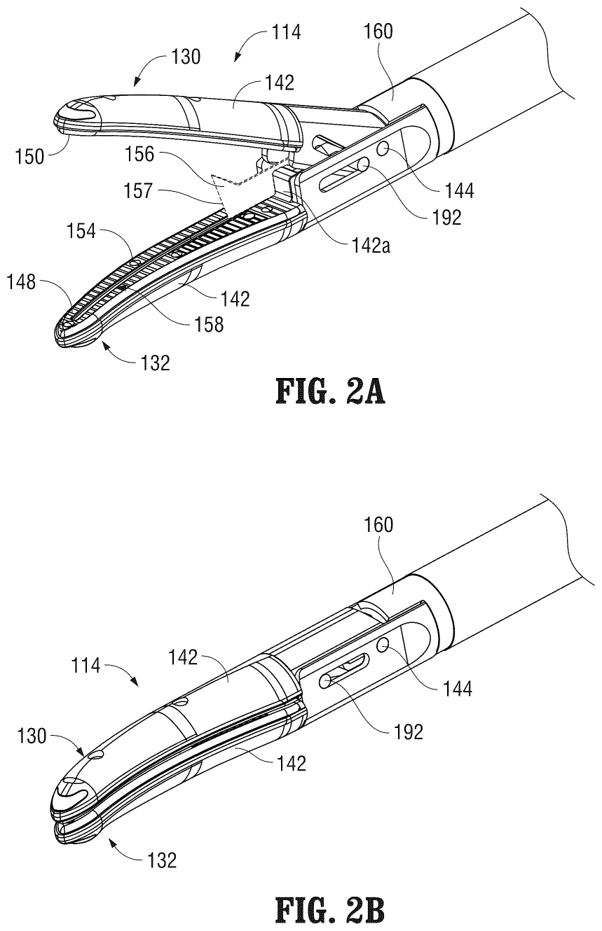

To mechanically control the end effector 114, the housing 112 supports a stationary handle 120, a movable handle 122, a trigger 126 and a rotation knob 128. The movable handle 122 is operable to move the end effector 114 between an open configuration (FIG. 2A) wherein a pair of opposed jaw members 130, 132 are disposed in spaced relation relative to one another, and a closed or clamping configuration (FIG. 2B) wherein the jaw members 130, 132 are closer together. Approximation of the movable handle 122 with the stationary handle 120 serves to move the end effector 114 to the closed configuration and separation of the movable handle 122 from the stationary handle 120 serves to move the end effector 114 to the open configuration. The trigger 126 is operable to extend and retract a knife blade 156 (see FIGS. 2A and 2B) through the end effector 114 when the end effector 114 is in the closed configuration. The rotation knob 128 serves to rotate the elongated shaft 116 and the end effector 114 about a longitudinal axis A-A extending through the forceps 114.

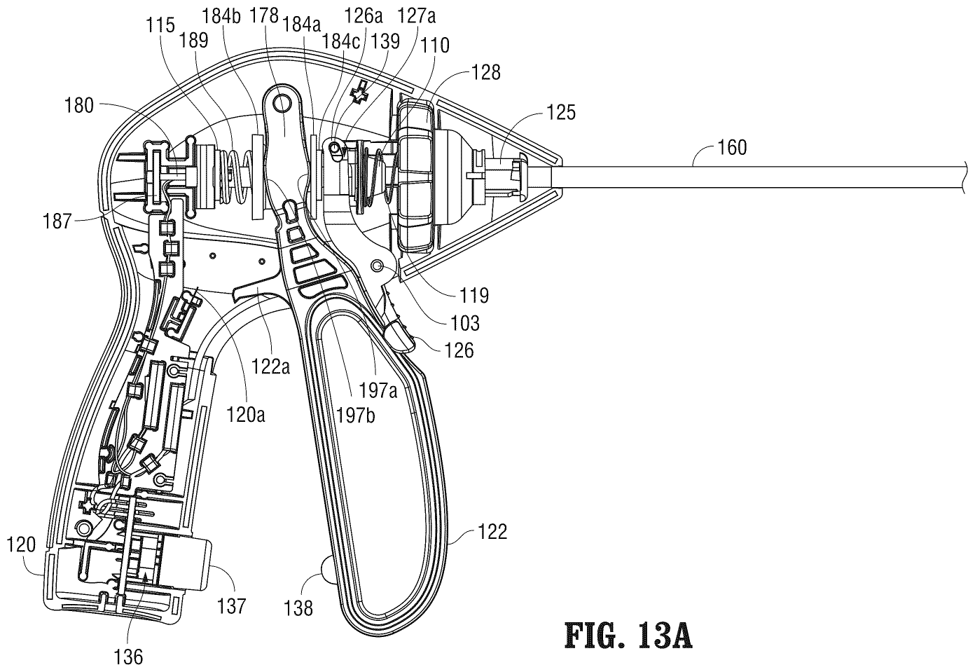

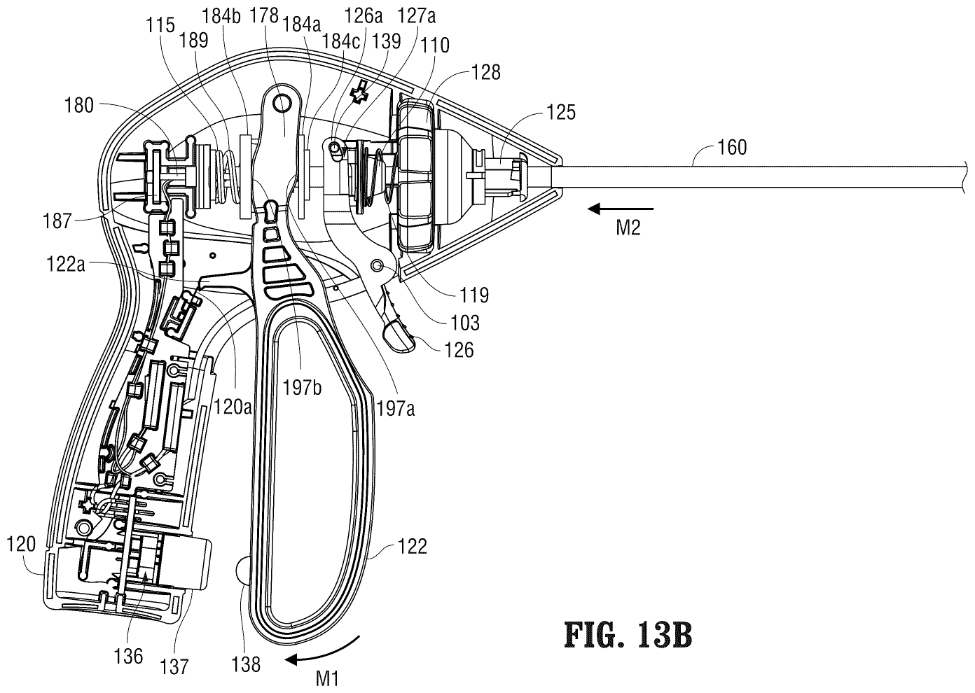

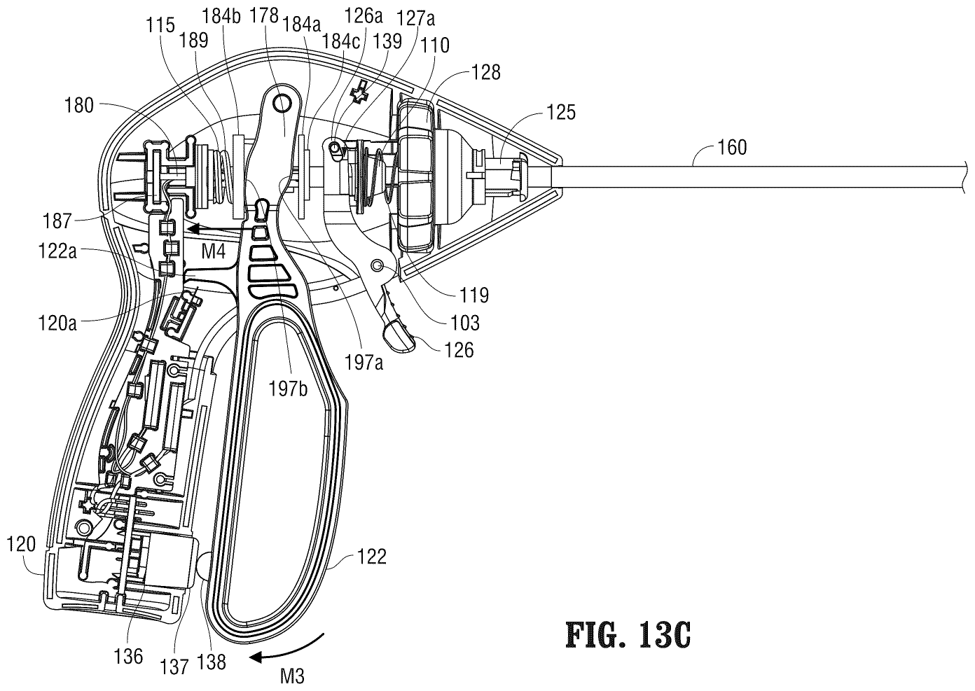

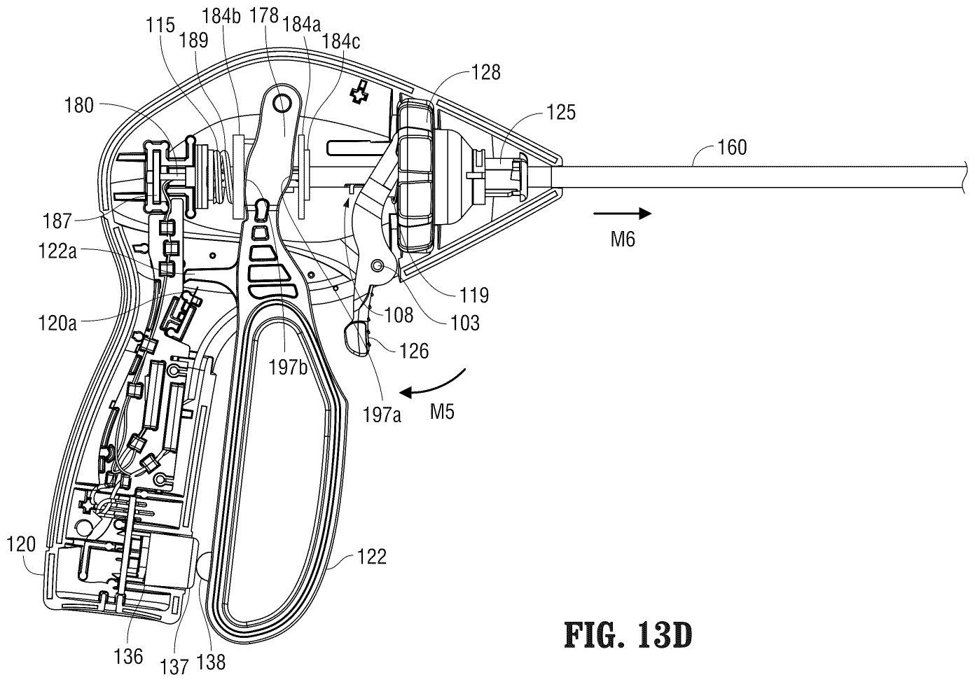

To electrically control the end effector 114, the stationary handle 120 supports a depressible button 137 thereon, which is operable by the user to initiate and terminate the delivery of electrosurgical energy to the end effector 114. The depressible button 137 is mechanically coupled to a switch 136 (FIGS. 13A-13D) disposed within the stationary handle 120 and is engageable by a button activation post 138 extending from a proximal side of the moveable handle 122 upon proximal movement of the moveable handle 122 to an actuated or proximal position (FIG. 13C). The switch 136 is in electrical communication with an electrosurgical generator 141 via suitable electrical wiring (not explicitly referenced) extending from the housing 112 through a cable 143 extending between the housing 112 and the electrosurgical generator 141. The generator 141 may include devices such as the LigaSure® Vessel Scaling Generator and the ForceTriad® Generator sold by Covidien. The cable 143 may include a connector (not shown) thereon such that the forceps 100 may be selectively coupled electrically to the generator 141.

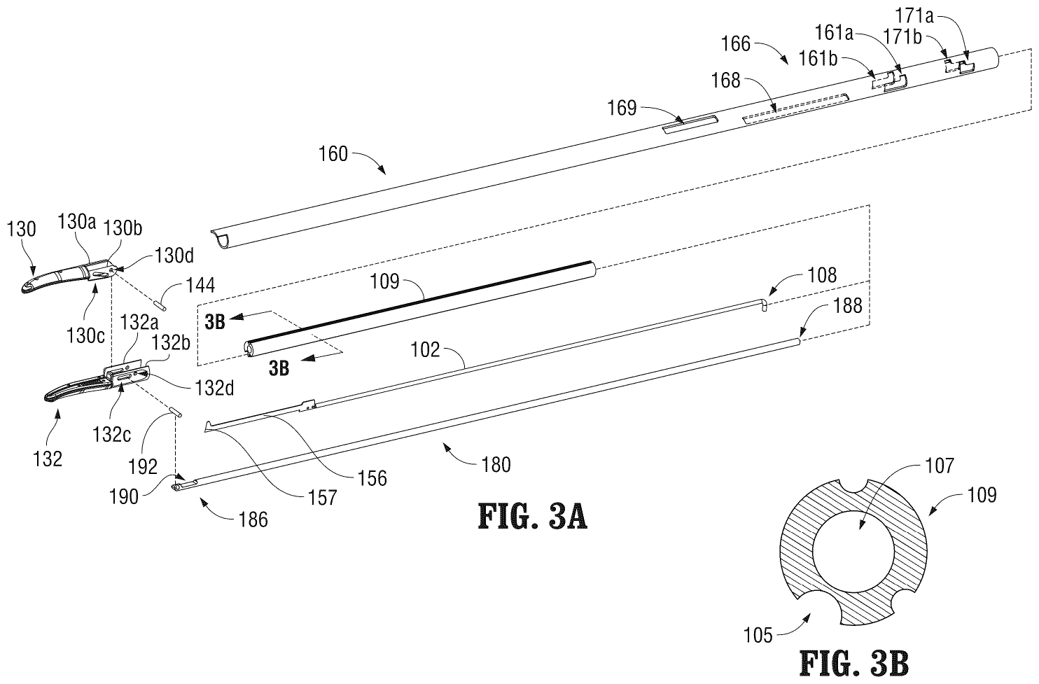

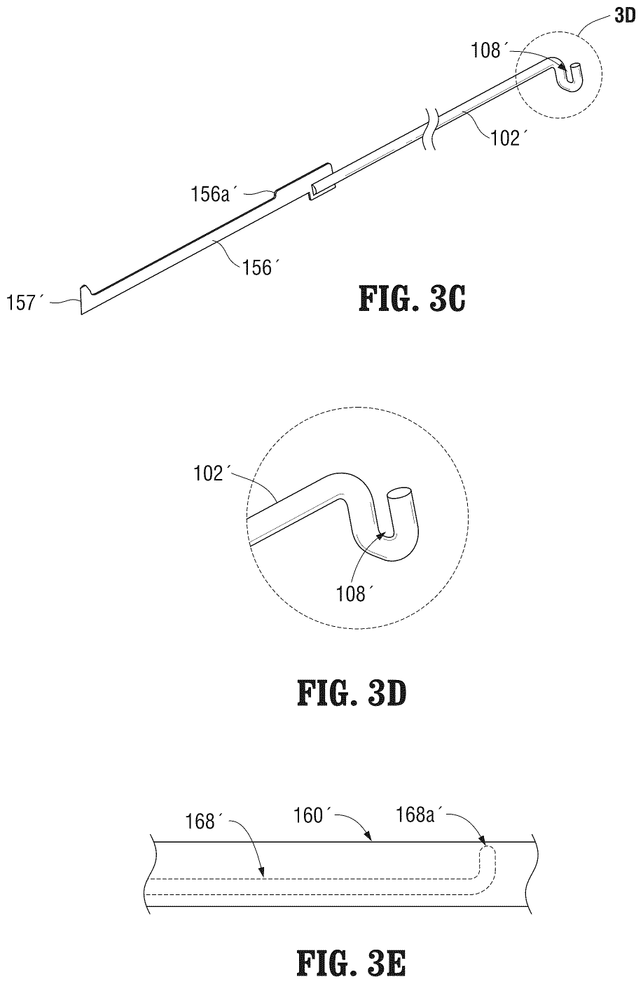

Referring now to FIGS. 2A-3A, the end effector 114 may be moved from the open configuration (FIG. 2A) wherein tissue (not shown) is received between the jaw members 130, 132, and the closed configuration (FIG. 2B), wherein the tissue is clamped and treated. The jaw members 130, 132 pivot about a pivot pin 144 to move the end effector 114 to the closed configuration of FIG. 2B wherein the sealing plates 148, 150 provide a pressure to tissue grasped therebetween. In some embodiments, to provide an effective tissue seal, a pressure within a range between about 3 kg/cm² to about 16 kg/cm² and, desirably, within a working range of about 7 kg/cm² to about 13 kg/cm², may be applied to the tissue. Also, in the closed configuration, a separation or gap distance is maintained between the sealing plates 148, 150 by an array of stop members 154 (FIG. 2A) disposed on or adjacent the sealing plates 148, 150. The stop members 154 contact opposing surfaces on the opposing jaw member 130, 132 and prohibit further approximation of the sealing plates 148, 150. In some embodiments, to provide an effective tissue seal, an appropriate gap distance of about 0.001 inches to about 0.010 inches and, desirably, between about 0.002 inches to about 0.005 inches, may be provided. In some embodiments, the stop members 154 are constructed of a heat-resistant ceramic deposited onto the jaw members 130, 132. In other embodiments, the stop members 154 are constructed of an electrically non-conductive plastic molded onto the jaw members 130, 132, e.g., by a process such as overmolding or injection molding. The stop members 154 may define any suitable number, arrangement, and/or configuration, depending on a particular purpose.

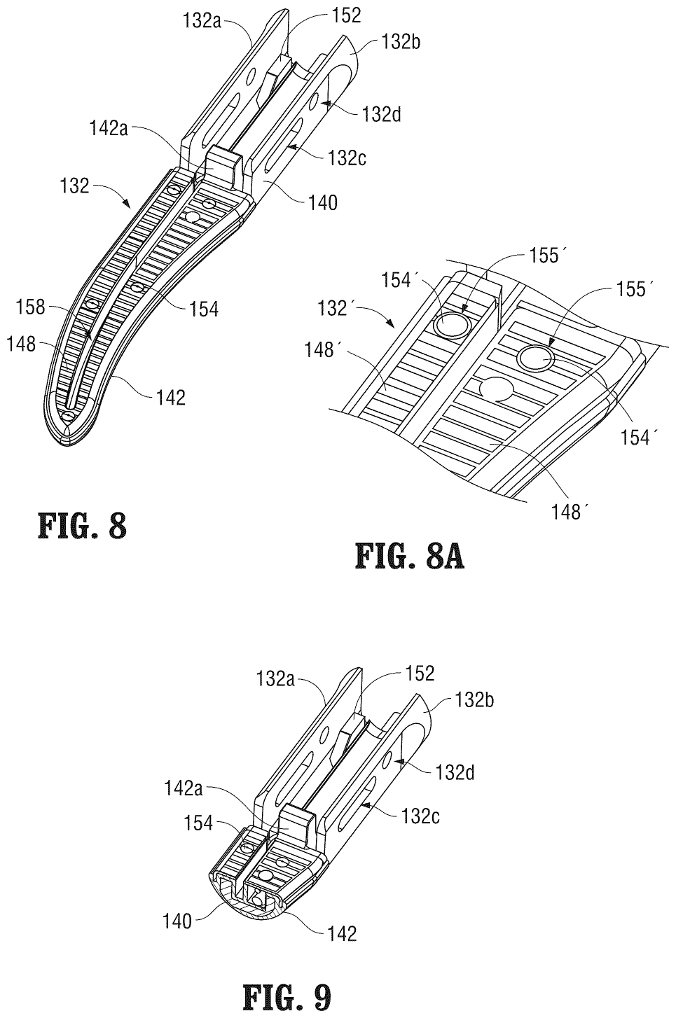

Referring momentarily to FIG. 8A, another embodiment of a lower jaw member 132′ is shown. Lower jaw member 132′ is similar to lower jaw member 132 (FIGS. 2A-3A) except as detailed below. Lower jaw member 132′ includes a sealing plate 148′ having a plurality of stop members 154′ disposed thereon in any suitable configuration. A wetting ring 155′ defined within the sealing plate 148′ is disposed about each of the stop members 154′. Wetting rings 155′ may be formed via etching or other suitable process and are formed on sealing plate 148′ prior to depositing (or otherwise forming) the stop members 154′. Upon depositing the ceramic onto sealing plate 148′ to form the stop members 154′ (or prior to otherwise forming the stop members 154′), wetting rings 155′ facilitate the formation of each of the stop members 154′ in a particular shape, e.g., circular, thus providing greater shape uniformity among the plurality of stop members 154′.

Referring again to FIGS. 2A-3A, upper and lower jaw members 130, 132 are electrically coupled to cable 143, and thus to the generator 141 (e.g., via respective suitable electrical wiring extending through the elongated shaft 116) to provide an electrical pathway to a pair of electrically conductive, tissue-engaging sealing plates 148, 150 disposed on the lower and upper jaw members 132, 130, respectively. The sealing plate 148 of the lower jaw member 132 opposes the sealing plate 150 of the upper jaw member 130. In some embodiments, the sealing plates 148 and 150 are electrically coupled to opposite terminals, e.g., positive or active (+) and negative or return (−) terminals associated with the generator 141. Thus, bipolar energy may be provided through the sealing plates 148 and 150 to tissue. Alternatively, the sealing plates 148 and 150 may be configured to deliver monopolar energy to tissue. In a monopolar configuration, one or both sealing plates 148 and 150 deliver electrosurgical energy from an active terminal, e.g., (+), while a return pad (not shown) is placed generally on a patient and provides a return path to the opposite terminal, e.g., (−), of the generator 141. Each jaw member 130, 132 includes a jaw insert 140 and an insulator 142 that serves to electrically insulate the sealing plates 150, 148 from the jaw insert 140 of the jaw members 130, 132, respectively.

Electrosurgical energy may be delivered to the tissue through the electrically conductive seal plates 148, 150 to effect a tissue seal. Once a tissue seal is established, a knife blade 156 having a sharpened distal edge 157 may be advanced through a knife channel 158 defined in one or both jaw members 130, 132 to transect the sealed tissue. Although the knife blade 156 is depicted in FIG. 2A as extending from the elongated shaft 116 when the end effector 114 is in an open configuration, in some embodiments, extension of the knife blade 156 into the knife channel 158 when the end effector 114 is in the open configuration is prevented, as discussed below with reference to FIGS. 13A-13D.

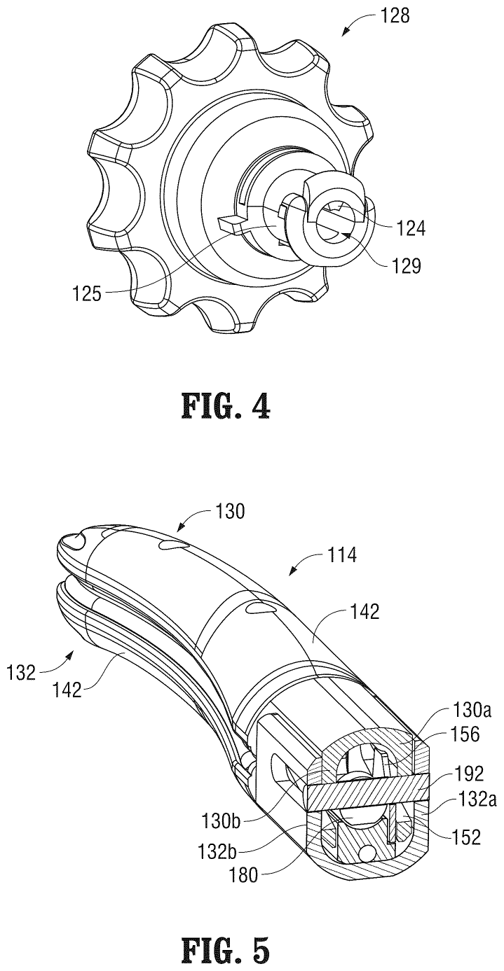

Referring to FIG. 3A, the elongated shaft 116 includes various longitudinal components that operatively couple the end effector 114 to the various actuators supported by the housing 112 (FIG. 1). An outer shaft member 160 defines an exterior surface of the elongated shaft 116 and houses other components therein as described below. The outer shaft member 160 is configured for longitudinal motion with respect to an inner actuation member 180 axially received within the outer shaft member 160. The inner actuation member 180 may be a rod, a shaft, a tube, folded metal, stamped metal, or other suitable structure. A proximal portion 166 of the outer shaft member 160 is configured for receipt within the housing 112 (FIG. 1), and includes features for operatively coupling the outer shaft member 160 to various elements of the housing 112. More specifically, the proximal portion 166 of the outer shaft member 160 includes, in order from distal to proximal, a longitudinal slot 169 to couple the outer shaft member 160 to the rotation knob 128, a longitudinal knife slot 168 defined therethrough, a pair of opposing distal locking slots 161a, 161b, and a pair of opposing proximal locking slots 171a, 171b. The connection established between the outer shaft member 160 and the rotation knob 128 is described below with reference to FIG. 4.

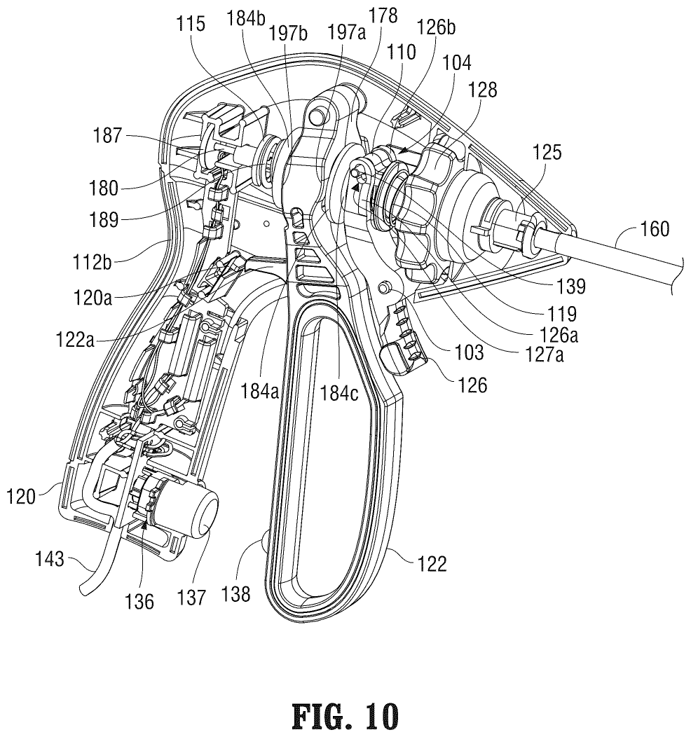

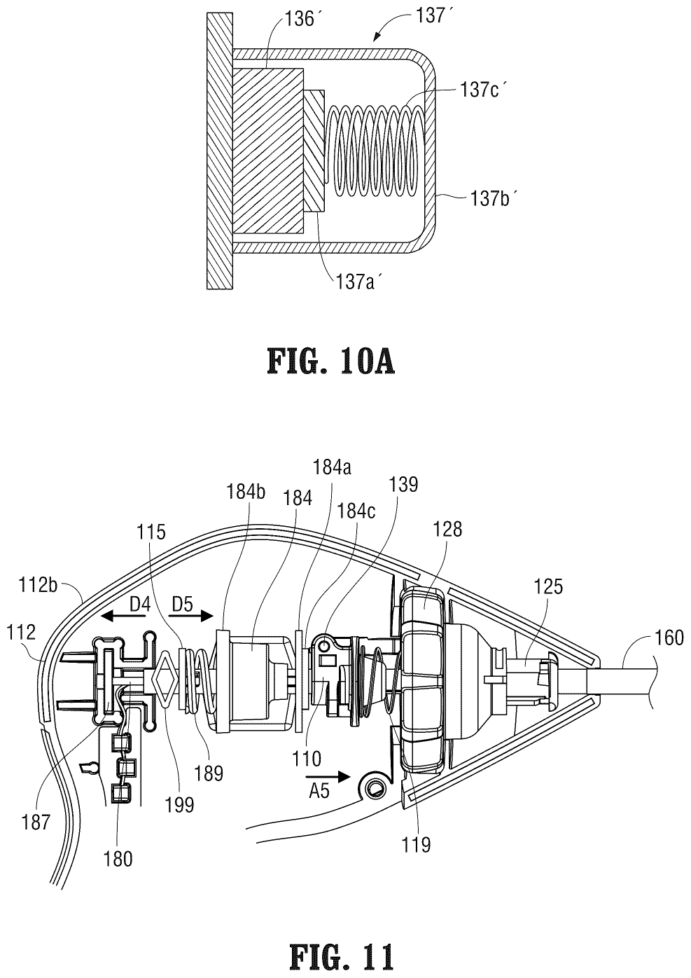

A distal portion 186 of the inner actuation member 180 includes a longitudinal recess 190 defined therein that provides clearance for the pivot pin 144 and thus, permits longitudinal reciprocation of the pivot pin 144 (via longitudinal reciprocation of the outer shaft member 160) independent of the inner actuation member 180. Distally of the longitudinal recess 190, a cam pin 192 is mechanically coupled (e.g., via welding, friction-fit, laser welding, etc) to the distal portion 186 of the inner actuation member 180. A proximal portion 188 of the inner actuation member 180 includes a washer 187 coupled thereto (FIG. 10). The washer 187 is captured within the housing 112 and serves to prohibit longitudinal motion of the inner actuation member 180 parallel to the longitudinal axis A-A.

The pivot pin 144 extends through a proximal portion of each of the jaw members 130, 132 to pivotally support the jaw members 130, 132 at the distal end of the inner actuation member 180. A proximal portion of each of the jaw members 130, 132 includes two laterally spaced parallel flanges or “flags” 130a, 130b and 132a, 132b respectively, extending proximally from a distal portion of the jaw members 130 and 132 (FIGS. 3A, 5, and 7-9). A lateral cam slot 130c and a lateral pivot bore 130d extend through each of the flags 130a, 130b of the upper jaw member 130 (FIG. 3A). Similarly, a lateral cam slot 132c and a lateral pivot bore 132d extend through each of the flags 132a, 132b of the lower jaw member 132 (FIGS. 8 and 9). The pivot bores 130d, 132d receive the pivot pin 144 in a slip-fit relation that permits the jaw members 130, 132 to pivot about the pivot pin 144 to move the end effector 114 between the open and closed configurations (FIGS. 2A and 2B, respectively).

A knife rod 102 is coupled (e.g., via welding) at a distal-most end to the sharpened knife blade 156 and includes an angled proximal end 108 that provides a mechanism for operatively coupling the knife rod 102 to the trigger 126. In some embodiments, the angled proximal end 108 of the knife rod 102 is formed by bending the knife rod 102 ninety degrees at its proximal end during manufacturing. The connection between the knife rod 102 and the trigger 126 is described in detail below with reference to FIGS. 10, 11, 12A, and 12B. The sharpened distal edge 157 of the knife blade 156 may be applied to the distal end of the knife blade 156 using a variety of manufacturing techniques such as, for example, grinding, coining, electrochemical etching, electropolishing, or other suitable manufacturing technique, for forming sharpened edges.

Referring to FIGS. 3A and 3B, a tube guide 109 is disposed within the outer shaft member 160 and includes a lumen 107 axially disposed therethrough. The inner actuation member 180 is received within the guide lumen 107, which serves to orient and align the inner actuation member 180 within the outer shaft member 160. The knife rod 102 is received within a longitudinal guide recess 105 formed in the outer surface of the guide tube 109. The guide recess 105 serves to guide longitudinal motion of the knife rod 102 within the outer shaft member 160 and to radially space the knife rod 102 from the inner actuation member 180 to prevent the inner actuation member 180 from interfering with reciprocal motion of the knife rod 102.

Referring now to FIG. 4, the rotation knob 128 includes a distal portion 125 extending distally therefrom and a passageway 129 defined therethrough for receiving the outer shaft member 160. The passageway 129 has a generally circular profile corresponding to the circular profile of the outer shaft member 160. The passageway 129 includes a longitudinal keying member 124 that is configured to align with and be seated within longitudinal slot 169 (FIG. 3A) of the outer shaft member 160. The keying member 124 projects laterally inward along the length of passageway 129 such that the insertion of the outer shaft member 160 into the passageway 129 of the rotation knob 128 operatively couples the outer shaft member 160 to the rotation knob 128. Rotational motion imparted to the rotation knob 128 may thus impart rotational motion to each of the components of the elongated shaft 116, and to the end effector 114, which is coupled thereto. As shown in FIGS. 10, 11, and 13A-13D, the rotation knob 128 is supported in the housing 112 and, as shown in FIG. 1, extends radially outward from opposing sides of the housing 112 (only shown extending radially outward from housing half 112b).

Referring now to FIG. 5, the end effector 114 is coupled to the distal end of the inner actuation member 180 by the cam pin 192. The cam pin 192 represents a longitudinally stationary reference for longitudinal movement of the outer shaft member 160 and the knife rod 102. The cam pin 192 extends through the flags 132a, 132b of the lower jaw member 132 and the flags 130a and 130b of the upper jaw member 130.

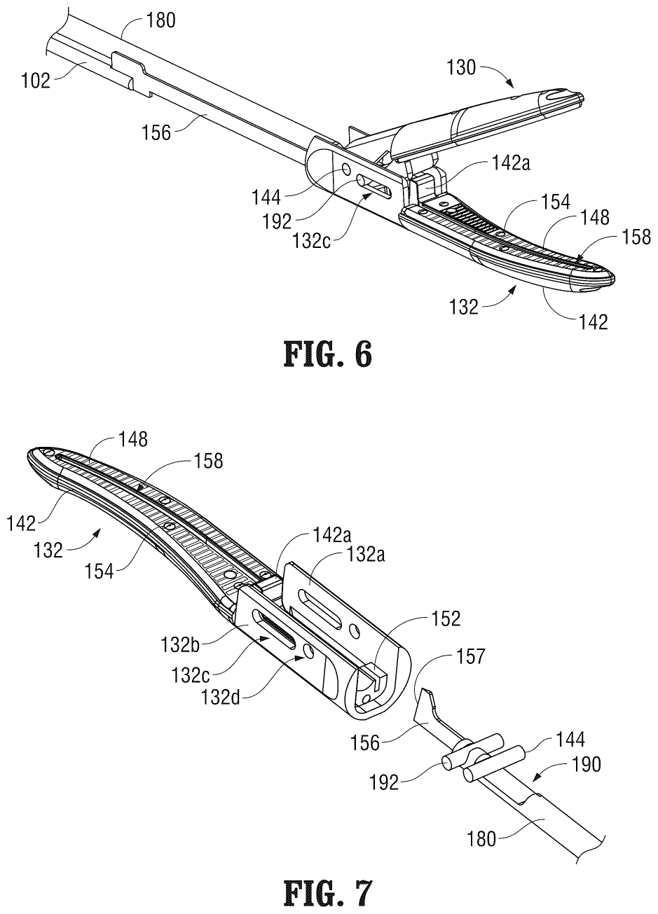

Referring now to FIG. 6, the end effector 114 is shown in the open configuration. Since the inner actuation member 180 is coupled to the cam pin 192, when the outer shaft member 160 (removed from view in FIG. 6 for clarity) is in an unactuated or distal position such that the inner actuation member 180 is in a proximal position relative to the outer shaft member 160, the cam pin 192 is located in a proximal position in cam slots 130c and 132c defined through the flags 130a, 130b, 132a, 132b of the jaw members 130, 132, respectively.

The outer shaft member 160 may be drawn proximally relative to the inner actuation member 180 and the cam pin 192 to move the end effector 114 to the closed configuration (see FIG. 2B). Since the longitudinal position of the cam pin 192 is fixed, and since the cam slot 130c is obliquely arranged with respect to the longitudinal axis A-A, proximal retraction of the outer shaft member 160 induces distal translation of the cam pin 192 through the cam slots 130c, 132c such that the jaw member 130 pivots toward jaw member 132 about the pivot pin 144. Conversely, when the end effector 114 is in the closed configuration, longitudinal translation of the outer shaft member 160 in a distal direction induces proximal translation of the cam pin 192 through the cam slots 130c, 132c such that jaw member 130 pivots away from jaw member 132 toward the open configuration.

In some embodiments, the inner actuation member 180 may be configured to move relative to the outer shaft member 160 to move the end effector 114 between the open and closed configurations. In this scenario, the moveable handle 122 may be operably coupled to the inner actuation member 180 and the washer 187 coupled to the proximal portion 188 of the inner actuation member 180 may be removed such that the inner shaft member 180 is free to move longitudinally along the longitudinal axis A-A upon actuation of the moveable handle 122. Proximal retraction of the inner actuation member 180 may induce proximal translation of the cam pin 192 through the cam slots 130c, 132c such that the jaw member 130 pivots away from jaw member 132 about the pivot pin 144 toward the open configuration. Conversely, when the end effector 114 is in the open configuration, longitudinal translation of the inner actuation member 180 in a distal direction induces distal translation of the cam pin 192 through the cam slots 130c, 132c such that jaw member 130 pivots toward jaw member 132 toward the closed configuration.

Referring now to FIG. 7, the pins 144, 192 do not interfere with the reciprocal motion of the knife blade 156. A proximal portion of the insulator 142 forms a blade guide 152 (also see FIGS. 5, 8, and 9) that serves to align the knife blade 156 such that the knife blade 156 readily enters the knife channel 158 defined in the jaw members 130, 132 (jaw member 130 removed from view in FIG. 7 for clarity).

Referring now to FIGS. 8 and 9, the lower jaw member 132 is constructed of three major components: the jaw insert 140, the insulator 142, and the sealing plate 148. The flags 132a, 132b of the jaw member 132 define a proximal portion of the jaw insert 140 and a generally u-shaped profile of the jaw insert 140 extends distally to support the tissue engaging portion of the jaw member 132. Upper jaw member 130 includes the same three major components as lower jaw member 132, including sealing plate 150, jaw insert 140, and insulator 142, and is constructed in the same manner as lower jaw member 132. However, lower jaw member 132 is fixedly engaged, e.g., welded, to outer shaft member 160, while upper jaw member 130 is pivotable relative to lower jaw member 132 and outer shaft member 160 between the open and closed configurations. In order to facilitate alignment of lower jaw member 132 and, more particularly, jaw insert 140 of lower jaw member 132, with outer shaft member 160 during welding (or other suitable fixed engagement), jaw insert 140 and outer shaft member 160 may include complementary alignment features, e.g., a complementary recess (not explicitly shown) defined within jaw insert 140 and a complementary protrusion (not explicitly shown) extending from outer shaft member 160. As an alternative to the unilateral configuration detailed above, both of the upper and lower jaw members 130, 132, respectively, may be pivotable relative to one another and outer shaft member 160, thus defining a bilateral configuration.

The insulator 142 of jaw members 130, 132 may be constructed of an electrically insulative plastic such as a polyphthalamide (PPA) (e.g., Amodel®), polycarbonate (PC), acrylonitrile butadiene styrene (ABS), a blend of PC and ABS, nylon, ceramic, etc. The insulator 142 may be overmolded onto the jaw insert 140 in either a single-shot or a two-shot injection molding process such that each of the sealing plates 148, 150 are coupled to and in spaced relation with their respective jaw inserts 140. Additionally or alternatively, the insulator 142 may be mechanically coupled to the jaw insert 140, e.g., pressed, snapped, glued, etc. Various features may be molded into the insulator 142 that facilitate the attachment of the sealing plates 148, 150 to the jaw inserts 140. For example, tabs may be provided that permit a snap-fit attachment, or ridges may be formed that permit ultrasonic welding of the sealing plates 148, 150 onto the insulators 142. In some embodiments, the insulator 142 on the lower jaw member 132 forms a tissue stop 142a extending therefrom adjacent to the knife channel 158 and proximal to the sealing plate 148. The tissue stop 142a serves to prevent tissue from entering the distal end of the outer shaft member 160 and to prevent splay of the flags 130a, 130b of the upper jaw member 130. In some embodiments, the tissue stop 142a may be formed by the insulator 142 on the upper jaw member 130 or on both the upper jaw member 130 and the lower jaw member 132. The tissue stop 142a may also serve to align the knife blade 156 as the knife blade 156 enters the knife channel 158 defined in the jaw members 130, 132. To this end, the surface of the tissue stop 142a extending along the path of the knife blade 156 may define a chamfered configuration to further facilitate alignment of the knife blade 156 as the knife blade 156 enters the knife channel 158.

Referring now to FIG. 10, the connection of the movable handle 122 and the knife trigger 126 to the longitudinally movable components of the elongated shaft 116 is described. The movable handle 122 may be manipulated to impart longitudinal motion to the outer shaft member 160, and the knife trigger 126 may be manipulated to impart longitudinal motion to the knife rod 102. As discussed above, longitudinal motion of the outer shaft member 160 serves to move the end effector 114 between the open configuration of FIG. 2A and the closed configuration of FIG. 2B, and longitudinal motion of the knife rod 102 serves to move knife blade 156 through knife channel 158 (FIG. 2A).

The movable handle 122 is operatively coupled to the outer shaft member 160 by a clevis 178 defined at an upper end of the movable handle 122. The clevis 178 is pivotally supported on the housing 112. The clevis 178 extends upwardly about opposing sides of a drive collar 184 (FIG. 11) supported on the outer shaft member 160 and includes rounded drive surfaces 197a and 197b thereon. Drive surface 197a engages a proximal-facing surface of a distal spring washer 184a and drive surface 197b engages a distal facing surface of a proximal rim 184b of the drive collar 184 (FIG. 11). The distal spring washer 184a engages a proximal facing surface of a distal spring stop 184c that, in turn, engages the opposing distal locking slots 161a, 161b (FIG. 3A) extending through the proximal portion 166 (FIG. 3A) of the outer shaft member 160 to couple the distal spring stop 184c to the outer shaft member 160. The drive surfaces 197a, 197b are arranged along the longitudinal axis A-A such that pivotal motion of the movable handle 122 induces corresponding longitudinal motion of the drive collar 184 (FIG. 11) along the longitudinal axis A-A.

Referring now to FIG. 11, proximal longitudinal motion may be imparted to the outer shaft member 160 by pushing the proximal rim 184b of the drive collar 184 proximally with the movable handle 122 (FIG. 10) as indicated by arrow D4 (FIG. 11). A spring 189 is constrained between a proximal facing surface of the drive collar 184 and a proximal spring stop 115. The proximal spring stop 115 engages the opposing proximal locking slots 171a, 171b (FIG. 3A) extending through the proximal portion 166 (FIG. 3A) of the outer shaft member 160 to couple the proximal spring stop 115 to the outer shaft member 160. Thus, the proximal spring stop 115 serves as a proximal stop against which spring 189 compresses.

Distal longitudinal motion is imparted to the outer shaft member 160 by driving the drive collar 184 distally with the movable handle 122 (FIG. 10). Distal longitudinal motion of the drive collar 184 induces a corresponding distal motion of the outer shaft member 160 by virtue of the coupling of the drive collar 184 to opposing distal locking slots 181a, 181b extending through the proximal portion 166 of the outer shaft member 160 (FIG. 3A). In some embodiments, a kick-out spring 199 is positioned between proximal spring stop 115 and a portion of housing 112 to ensure full return of outer shaft member 160 distally upon release or return of movable handle 122 (FIG. 10). The kick-out spring 199 may include a pair of plate surfaces interconnected via living hinges, as shown, although any other suitable spring may be provided.

Proximal longitudinal motion of the outer shaft member 160 draws jaw member 132 proximally such that the cam pin 192 advances distally to pivot jaw member 130 toward jaw member 132 to move the end effector 114 to the closed configuration as described above with reference to FIG. 6. Once the jaw members 130 and 132 are closed, the outer shaft member 160 essentially bottoms out (i.e., further proximal movement of the outer shaft member 160 is prohibited since the jaw members 130, 132 contact one another). Further proximal movement of the movable handle 122 (FIG. 10), however, will continue to move the drive collar 184 proximally. This continued proximal movement of the drive collar 184 further compresses the spring 189 to impart additional force to the outer shaft member 160, which results in additional closure force applied to tissue grasped between the jaw members 130, 132 (see FIG. 2B).

Referring again to FIG. 10, the trigger 126 is pivotally supported in the housing 112 about a pivot boss 103 protruding from the trigger 126. The trigger 126 is operatively coupled to the knife rod 102 by a knife connection mechanism 104 such that pivotal motion of the trigger 126 induces longitudinal motion of the knife rod 102. The knife connection mechanism 104 includes upper flanges 126a, 126b of the trigger 126 and a knife collar 110.

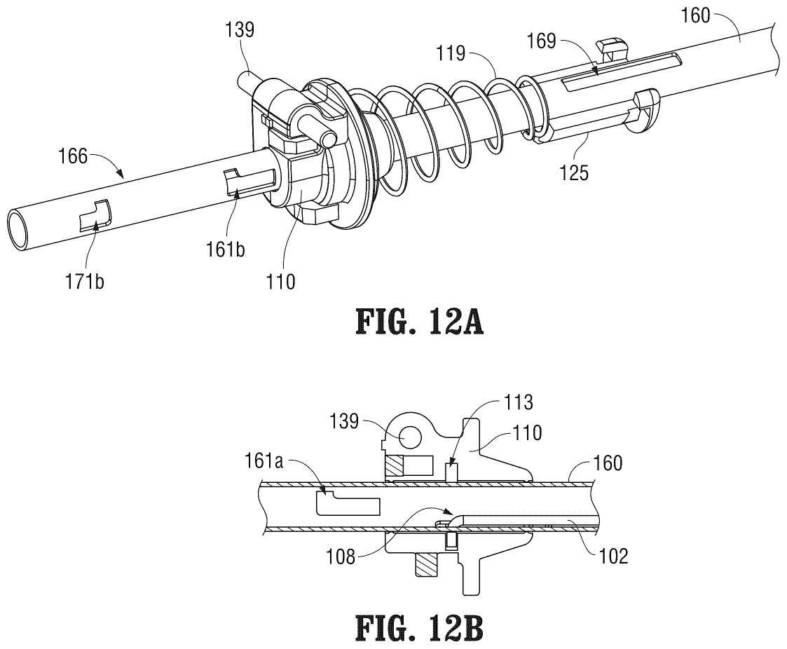

Referring now to FIGS. 11, 12A, and 12B, the knife collar 110 includes a pair of integrally formed pin bosses 139a, 139b extending from opposing sides thereof. As shown by FIG. 12B, the knife collar 110 includes an interior circular channel 113 that captures the angled proximal end 108 of the knife rod 102 to couple the knife rod 102 to the knife collar 110. Referring momentarily to FIGS. 3C and 3D, in conjunction with FIG. 12B, in some embodiments, the proximal end 108′ of the knife rod 102′ may alternatively define a hooked configuration to help further inhibit disengagement of the proximal end 108′ of the knife rod 102′ from within the channel 113 of the knife collar 110. In such embodiments, as shown in FIG. 3E, the knife slot 168′ defined within the outer shaft member 160′ further includes an angled portion 168a′ disposed at the proximal end thereof to accommodate the hooked proximal end 108′ of the knife rod 102′.

Referring again to FIGS. 11, 12A, and 12B, longitudinal motion of the outer shaft member 160, the angled proximal end 108 of the knife rod 102 translates longitudinally within knife slot 168 (FIG. 3A) of the outer shaft member 160 such that the longitudinal motion of outer shaft member 160 is unimpeded by the angled proximal end 108 of the knife rod 102. Upon rotation of the elongated shaft 116 and end effector 114 about the longitudinal axis A-A via the rotation knob 128 (FIG. 1), the angled proximal end 108 of the knife rod 102 freely rotates within the interior circular channel 113 of the knife collar 110 such that the outer and inner actuation members 160 and 180 (removed from view in FIG. 12B for clarity), and the knife rod 102 rotate within the knife collar 110 about the longitudinal axis A-A. In this way, the knife collar 110 serves as a stationary reference for the rotational movement of the outer shaft member 160, the inner actuation member 180, and the knife rod 102.

Referring again to FIG. 10, the upper flanges 126a, 126b of the trigger 126 include respective slots 127a, 127b defined therethrough that are configured to receive the pin bosses 139a, 139b, respectively, of the knife collar 110 such that pivotal motion of the trigger 126 induces longitudinal motion of the knife collar 110 and, thus, the knife rod 102 by virtue of the coupling of knife rod 102 to the knife collar 110.

Referring now to FIGS. 11 and 12A, when the trigger 126 is moved to induce motion of the knife collar 110 in order to translate the blade 156 through the knife channel 158, the knife collar 110 translates along the outer shaft member 160 in the direction of arrow A5 to abut a spring 119 such that spring 119 compresses against the distal portion 125 of the rotation knob 128 (FIG. 12A). The spring 119 biases the knife collar 110 proximally along the outer shaft member 160. With reference to FIG. 3C, in some embodiments, a hard stop 156a′ formed at the proximal end of knife blade 156′ is provided for interference with the pivot pin 144 of end effector 114 (FIG. 2B) to limit the travel distance, e.g., extension, of knife blade 156′.

Referring now to FIGS. 13A, 13B, 13C and 13D, a sequence of motions may be initiated by moving the movable handle 122 to induce motion of the outer shaft member 160 in order to close the jaws 130, 132, and by moving the trigger 126 to induce motion of the knife collar 110 in order to translate the blade 156 through the knife channel 158. Initially, both the moveable handle 122 and the knife trigger 126 are in a distal or un-actuated position as depicted in FIG. 13A. This arrangement of the moveable handle 122 and trigger 126 sustains the end effector 114 in the open configuration (FIG. 2A) wherein the jaw members 130, 132 are substantially spaced from one another, and the knife blade 156 is in a retracted or proximal position with respect to the jaw members 130, 132. When both the moveable handle 122 and the knife trigger 126 are in the distal, un-actuated position, pivotal motion of the knife trigger 126 in a proximal direction, i.e., toward the stationary handle 120, is passively prohibited by interference between the trigger 126 and moveable handle 122. This interference prohibits advancement of the knife blade 156 through the knife channel 158 when the end effector 114 is in the open configuration. Movable handle 122 may additionally include a protrusion (not shown) or other feature extending distally therefrom that is configured to contact the trigger 126 upon return of the movable handle 122 distally towards the un-actuated position, thereby returning the trigger 126 towards its un-actuated position if not previously returned via the spring 119 (FIG. 11).

The movable handle 122 may be moved from the distal position of FIG. 13A to the intermediate position depicted in FIG. 13B to move the jaw members 130, 132 to the closed configuration (FIG. 2B). As the movable handle 122 pivots in the direction of arrow M1 (FIG. 13B), the drive surface 197b of the movable handle 122 engages the proximal rim 184b of the drive collar 184. The drive collar 184 is driven proximally such that the spring 189 biases the proximal spring stop 115 and, thus, the outer shaft member 160 is driven proximally in the direction of arrow M2 (FIG. 13B). As discussed above with reference to FIG. 6, proximal movement of the outer shaft member 160 serves to translate the cam pin 192 distally though the cam slots 130c, 132c (FIG. 3A) of the jaw members 130, 132, respectively, and thus pivot jaw member 130 toward jaw member 132 (FIG. 2B). As the jaw members 130, 132 engage one another and no further pivotal movement of the jaw members 130, 132 may be achieved, further distal movement of the cam pin 192 and further proximal movement of the outer shaft member 160 are prevented.

As the movable handle 122 is moved from the distal position of FIG. 13A to the intermediate position depicted in FIG. 13B, a tooth 122a extending proximally from an upper portion of the moveable handle 122 engages a clicker tab 120a supported within the stationary handle 120 to generate a tactile and/or an audible response. The clicker tab 120a may be constructed of a plastic film, sheet metal, or any suitable material configured to generate a “clicking” sound as the clicker tab 120a is engaged and disengaged by the tooth 122a. This response generated by the clicker tab 120a corresponds to a complete grasping of tissue between the jaw members 130, 132 and serves to indicate to the surgeon that further pivotal motion of the moveable handle 122 in a proximal direction, i.e., toward the stationary handle 120, will cause the button activation post 138 to engage the depressible button 137. As the moveable handle 122 is moved from the intermediate position of FIG. 13B to the actuated or proximal position of FIG. 13C, the button activation post 138 depresses the depressible button 137, thereby activating the switch 136 disposed within the stationary handle 120 to initiate the delivery of electrosurgical energy to the end effector 114 to generate a tissue seal.

With reference to FIG. 10A, in some embodiments, the depressible button 137′ includes an inner button member 137a′ operably coupled to the switch 136′, an outer button member 137b′ disposed about the inner button member 137a′, and a spring 137c′ disposed between the inner and outer button members 137a′, 137b′, respectively. In order to activate the switch 136′ in such a configuration, the moveable handle 122 (FIG. 10) is moved to the actuated position such that the button activation post 138 (FIG. 10) depresses the outer button member 137b′ which, in turn, compresses and urges the spring 137c into contact with the inner button member 137a′ to depress the inner button member 137a′ and activate the switch 136′. This configuration is advantageous at least in that the biasing force of spring 137c′ maintains the switch 136′ in an activated state even if handle 122 (FIG. 10) is returned slightly, e.g., moved distally a slight distance. Thus, accidental release of some of the pressure on handle 122 (FIG. 10) will not deactivate the switch 136′.

Referring again to FIGS. 13A, 13B, 13C and 13D, the movable handle 122 is moved from the intermediate position of FIG. 13B to the actuated or proximal position of FIG. 13C, the pressure applied by the jaw members 130, 132 is increased. As the movable handle 122 pivots further in the direction of arrow M3 (FIG. 13C), the drive surface 197b presses the proximal rim 184b of the drive collar 184 further proximally against the spring 189 in the direction of arrow M4 (FIG. 13C). The spring 189 is compressed against the proximal spring stop 115, and a tensile force is transmitted through the outer shaft member 160 to the jaw members 130, 132. The tensile force supplied by the spring 189 ensures that the jaw members 130, 132 apply an appropriate pressure to effect a tissue seal.

When the movable handle 122 is in the actuated or proximal position, the knife trigger 126 may be selectively moved from the distal position of FIG. 13C to the proximal position of FIG. 13D to advance the knife blade 156 distally through knife channel 158. The knife trigger 126 may be pivoted in the direction of arrow M5 (FIG. 13D), about pivot boss 103 to advance the flanges 126a, 126b of the knife trigger 126 distally in the direction of arrow M6 such that the pin bosses 139a, 139b translate within respective slots 127a, 127b from the position shown in FIGS. 13A-13C to the position shown in FIG. 13D (flange 126b, pin boss 139b, and slot 127b are obstructed from view in FIGS. 13A-13D). Movement of flanges 126a, 126b draws the knife collar 110 distally, which induces distal longitudinal motion of the knife rod 102 by virtue of the coupling of the knife rod 102 to the knife collar 110, as described above with reference to FIG. 12B.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as examples of particular embodiments. Those skilled in the art will envision other modifications within the scope and spirit of the claims appended hereto.

Although the foregoing disclosure has been described in some detail by way of illustration and example, for purposes of clarity or understanding, it will be obvious that certain changes and modifications may be practiced within the scope of the appended claims.

1-18. (canceled)

19. A surgical instrument, comprising:
an outer shaft extending from a housing and defining a longitudinal axis;
an inner shaft disposed within the outer shaft;
a pair of opposing jaw members disposed at a distal end of the outer shaft, the jaw members configured to transition between an open configuration and a closed configuration for grasping tissue, each of the jaw members including a cam slot;
a pivot pin disposed through each of the jaw members, at least one of the jaw members configured to pivot about the pivot pin relative to the other jaw member to transition the jaw members between the open and closed configurations; and
a cam pin attached to a distal end portion of the inner shaft and disposed within the cam slot of each jaw member, wherein the pivot pin is configured to move along the longitudinal axis relative to the cam pin during transition of the jaw members between the open and closed configurations.

20. The surgical instrument according to claim 19, wherein movement of the outer shaft relative to the inner shaft causes movement of the pivot pin along the longitudinal axis relative to the cam pin during transition of the jaw members between the open and closed configurations.

21. The surgical instrument according to claim 19, wherein movement of the outer shaft relative to the inner shaft causes movement of the cam slot relative to the cam pin during transition of the jaw members between the open and closed configurations.

22. The surgical instrument according to claim 19, wherein movement of the outer shaft relative to the inner shaft causes corresponding movement of one of the jaw members along the longitudinal axis to move the cam slot relative to the cam pin during transition of the jaw members between the open and closed configurations.

23. The surgical instrument according to claim 19, wherein each of the jaw members includes an electrically conductive tissue sealing surface configured to electrically couple with a source of electrosurgical energy.

24. The surgical instrument according to claim 19, further comprising a knife blade configured to cut the tissue grasped between the jaw members.

25. The surgical instrument according to claim 24, wherein each of the jaw members includes a knife channel through which the knife blade moves to cut the tissue grasped between the jaw members.

26. The surgical instrument according to claim 19, wherein the inner shaft includes a longitudinal recess and the pivot pin is configured to move within the longitudinal recess during movement of the pivot pin along the longitudinal axis relative to the cam pin.

27. A surgical instrument, comprising:
an outer shaft extending from a housing and defining a longitudinal axis;
an inner shaft disposed within the outer shaft;
an end effector disposed at a distal end of the outer shaft and including a cam slot, the end effector configured to treat tissue;
a pivot pin disposed through the end effector, wherein at least a portion of the end effector is configured to pivot about the pivot pin; and
a cam pin attached to a distal end portion of the inner shaft and disposed within the cam slot, wherein the pivot pin is configured to move along the longitudinal axis relative to the cam pin during actuation of the end effector.

28. The surgical instrument according to claim 27, wherein movement of the outer shaft relative to the inner shaft causes movement of the pivot pin along the longitudinal axis relative to the cam pin during actuation of the end effector.

29. The surgical instrument according to claim 27, wherein movement of the outer shaft relative to the inner shaft causes movement of the cam slot relative to the cam pin during actuation of the end effector.

30. The surgical instrument according to claim 27, wherein the end effector includes a pair of opposing jaw members configured to transition between an open configuration and a closed configuration to grasp the tissue.

31. The surgical instrument according to claim 30, wherein movement of the outer shaft relative to the inner shaft causes corresponding movement of at least one of the jaw members along the longitudinal axis to move the cam slot relative to the cam pin during transition of the jaw members between the open and closed configurations.

32. The surgical instrument according to claim 27 further comprising a knife blade configured to cut the tissue.

33. The surgical instrument according to claim 32, wherein the end effector includes a knife channel through which the knife blade moves to cut the tissue.

34. The surgical instrument according to claim 27, wherein the inner shaft includes a longitudinal recess and the pivot pin is configured to move within the longitudinal recess during movement of the pivot pin along the longitudinal axis relative to the cam pin.

35. A surgical instrument, comprising:
an elongated shaft extending from a housing;
a pair of opposing jaw members disposed at a distal end of the elongated shaft, the pair of opposing jaw members configured to transition between an open configuration and a closed configuration for grasping tissue, each of the jaw members including a cam slot;
a pivot pin disposed through each of the jaw members, at least one of the jaw members configured to pivot about the pivot pin relative to the other jaw member to transition the jaw members between the open and closed configurations; and
a cam pin disposed within the cam slot of each jaw member, wherein the pivot pin is configured to move along the longitudinal axis relative to the cam pin during transition of the jaw members between the open and closed configurations.

36. The surgical instrument according to claim 35, further comprising an inner shaft disposed within the elongated shaft, wherein movement of the elongated shaft relative to the inner shaft causes movement of the pivot pin along the longitudinal axis relative to the cam pin during transition of the jaw members between the open and closed configurations.

37. The surgical instrument according to claim 36, wherein movement of the elongated shaft relative to the inner shaft causes movement of the cam slot relative to the cam pin during transition of the jaw members between the open and closed configurations.

38. The surgical instrument according to claim 36, wherein movement of the elongated shaft relative to the inner shaft causes corresponding movement of one of the jaw members along the longitudinal axis to move the cam slot relative to the cam pin during transition of the jaw members between the open and closed configurations.
